(19) United States
(12) Patent Application Publication
(10) Pub. No.: US 2013/0116546 A1
(43) Pub. Date: May 9, 2013

(54) METHOD AND MAGNETIC RESONANCE DEVICE TO ASSIST A PERSON CONDUCTING A MINIMALLY INVASIVE PROCEDURE

(71) Applicants: Martin Requardt, Nuernberg (DE); Eva Rothgang, Nuernberg (DE)

(72) Inventors: Martin Requardt, Nuernberg (DE); Eva Rothgang, Nuernberg (DE)

(21) Appl. No.: 13/661,273

(22) Filed: Oct. 26, 2012

(30) Foreign Application Priority Data
Oct. 27, 2011 (DE) 102011085308.1

(51) Int. Cl. A61B 5/055; A61B 5/06; A61B 5/00; A61B 17/00; A61B 19/00

(52) U.S. Cl. CPC A61B 5/055; A61B 17/00234; A61B 19/20; A61B 5/72; A61B 5/7405; A61B 5/062; A61B 5/064
USPC 600/414; 600/417

(57) ABSTRACT

In a method and magnetic resonance apparatus to assist a person in the alignment of a medical instrument used by the person to conduct a minimally invasive procedure with the instrument introduced into a patient at an entrance point in a predetermined orientation, within the patient receptacle of a magnetic resonance device, measurement data are acquired with the magnetic resonance device to determine a current orientation of the instrument and/or an instrument guide, and the measurement data are evaluated to determine the current orientation. A deviation of the current orientation from the predetermined orientation is determined, and at least one acoustic signal is emitted depending on the deviation to assist the person in orienting the instrument.

### BACKGROUND OF THE INVENTION

1. Field of the Invention

The invention concerns a method to assist a person who is conducting a minimally invasive procedure with an instrument (in particular a medical needle) to be introduced into a patient at an entrance point in a predetermined orientation, within the patient receptacle of a magnetic resonance device. More specifically the method concerns assisting such a person in the alignment of the instrument. The invention also concerns a magnetic resonance device for implementing such a method.

2. Description of the Prior Art

Minimally invasive procedures today are implemented with magnetic resonance assistance, including known minimally invasive procedures in which an instrument is to be introduced into the patient within the patient receptacle of a magnetic resonance device. Both a previously planned procedure point and a previously planned, predetermined orientation of the instrument must be adhered to as exactly as possible in order to achieve success of the minimally invasive procedure. Medical needles are frequently used as instruments for such a minimally invasive procedure, in particular examination and/or treatment needles (for example, biopsy needles or ablation needles).

In order to assist the person implementing the procedure in the alignment of the instrument, various solutions have been proposed, but they are all very complicated and expensive. For example, an additional (secondary) monitor—which monitor is also visible in the patient receptacle—can be provided within the examination room in which the magnetic resonance device is located, on which additional monitor is shown a visual feedback of magnetic resonance exposures acquired during the alignment of the instrument or feedback from a navigation system, and the alignment takes place using the information presented on the additional monitor. This requires special and very expensive hardware and simultaneously takes the attention of the person conducing the procedure away from the patient, to be directed toward the additional monitor.

It was also proposed to use robotic devices (a robot arm, for example) for the placement of instruments within the patient receptacle of a magnetic resonance device. Such solutions must be developed specifically for the magnetic resonance environment and are therefore complicated and expensive. In addition, robotic devices are less welcome to many physicians who wish to conduct the procedure based on their own experience.

Instrument guides that assist in the manual positioning or alignment of a patient are known. A particularly advantageous embodiment is distributed under the name “SeeStar” and is described in detail in WO 2004/021898 A1. The guide device described therein for a medical instrument is characterized by the adjustment to the predetermined entrance point being made only once, because any change of the orientation of the instrument guide, or the instrument arranged therein, does not modify the set entrance point. After an alignment to the predetermined orientation of the instrument, this alignment can likewise be fixed by means of an establishment device, which means that the instrument guide can be fixed so that a set, predetermined entrance point (and later a set orientation) are maintained.

### SUMMARY OF THE INVENTION

An object of the present invention is to provide an assistance method for alignment of an instrument within the patient receptacle of a magnetic resonance device that is realized with little effort as well as economically, and with which the attention of the physician can remain on the patient.

This object is achieved in a method according to the invention wherein measurement data are acquired with the magnetic resonance device to determine a current orientation of the instrument and/or an instrument guide, and the measurement data are evaluated in a processor to determine the current orientation, and a deviation of the current orientation from the predetermined orientation is determined by the processor and, to assist the attending physician, at least one acoustic signal is emitted that is dependent on the deviation.

The completely automatic method according to the invention, for example implemented by a control device of the magnetic resonance device, provides an acoustic guidance of the person conducting the procedure toward the predetermined orientation. Exclusively (only) acoustic signals are thus emitted so that a visual feedback (for example in the form of a complicated and expensive secondary monitor) is thus not necessary. MR-compatible display devices arranged in the room of the magnetic resonance device are consequently not absolutely necessary. The person conducting the procedure does not need to turn his or her attention away from the patient (at least during the adjustment of the orientation) in order to look at an additional display, because the acoustic signals can be perceived even while giving full concentration to the procedure region, in particular the entrance point and the instrument. Within the scope of the invention, however, it is nevertheless possible to also provide and use a display device to provide additional information to the person conducing the procedure, for example when an ideal entrance point should first be found under real-time imaging.

Additional advantages of the procedure according to the invention are that a target-oriented navigation is possible without the necessity of additional hardware or specially designed visualization platforms. Since use of additional hardware is avoided, additional sterilization steps are not needed.

In a preferred embodiment of the invention, the acoustic signal is emitted by a component of the magnetic resonance device. No additionally provided acoustic output emitter is necessary, and thus no new hardware is required in order to execute the method according to the invention, which can then be implemented solely by the magnetic resonance device, in particular by operation of the control device thereof. The aforementioned component includes an acoustic output emitter for communication with a patient, or preferably a gradient coil arrangement of the magnetic resonance device. A gradient coil arrangement can be particularly advantageously activated so that the background noise generated anyway by this arrangement is adapted so that the acoustic signal is created. Gradient coils have an acoustic coupling to additional structure components of the magnetic resonance device, in particular to casing (housing) parts, such that (normally unwanted) noises arise corresponding to the activation of the gradient coils. If the activation of the gradient coils is consequently modified, for example by adding additional gradient pulses in acquisition pauses, a variety of noises (and consequently acoustic signals) can be produced. The gradient coil arrangement (together with structural components of the magnetic resonance device) then ultimately acts as a form of acoustic speaker. The modulation of the gradient noise consequently allows a simple communication with the person conducting the procedure. For example, by modulation of the gradient pulses a communication similar to parking-assist systems in motor vehicles can be realized, such that the necessity of additional devices/hardware is avoided. In a specific, exemplary embodiment of the present invention, the acoustic signal is generated depending on the deviation by correspondingly changing a repetition time is selected in the activation of the gradient coil arrangement. The acoustic noises of the gradient coil arrangement thus can be modified to create the acoustic signal, for example, in that the repetition time (TR) being varied so that lower or higher pitches (tone frequencies) can be generated depending on the deviation from the predefined instrument path. For example, if repetition times in the range from 1-5 ms are used, frequencies in the range from 200-1000 Hz can be achieved. For example, the person conducting the procedure should then seek to achieve the highest possible tone. The tone frequency/the pitch of the tone that is emitted by the gradient coil arrangement as an acoustic signal consequently rises with decreasing deviation. Naturally, however, other activations of the gradient coil arrangement to emit an acoustic signal are also possible, for example to emit an uninterrupted acoustic signal, in particular a tone repeating with a specific repetition frequency, and the like.

Alternatively, an acoustic output emitted that is present anyway as part of the magnetic resonance device can be used, for example a speaker that is conventionally provided in the patient receptacle for communication with the patient, and/or headphones that, for example, are connected to a communication device that likewise serves for communication with the patient. For example, headphones for the patient and additional headphones for the person conducting the procedure can be provided via a splitter. Such an acoustic output emitter provided for communication with a patient allows a finer gradation of the acoustic signals as well as more differentiated acoustic signals. The acoustic signals can thus immediately be brought to the attention of the patient as well, but embodiments and situations are also conceivable in which the patient does not hear the acoustic signals, for example if the patient uses earplugs or the like, so the person conducting the procedure can use the acoustic output emitter alone.

To transmit signals that will produce the acoustic signals in the headphones, an optical magnetic resonance communication system is suitable, as is commercially available under the name “IMROC” from the company Optoacoustics Ltd., Israel, for example. This embodiment avoids the patient and the person conducting the procedure from being excessively subjected to the noise of the magnetic resonance device, and a simplified communication is enabled by adaptive noise reduction in the headphones.

In a further embodiment of the present invention, as discussed with regard to the use of the gradient coil arrangement as an output emitter for the acoustic signal, a tone with a repetition frequency depending on the deviation and/or a pitch depending on the deviation is produced as the acoustic signal. In this way a signal that is simple to interpret is achieved without complex instructions needing to be incorporated in the acoustic signal. The person conducting the procedure can intuitively coordinate the changing signal with a change of the instrument the orientation, without needing to take attention away from the procedure area at the patient.

Specifically, a tone with a pitch dependent on the deviation can be used as an acoustic signal. Given the use of a gradient coil arrangement of the magnetic resonance device to emit the acoustic signal, wherein a tone with a highest or lowest pitch is achieved in the case of a deviation of zero. For example, the pitch of the tone (and consequently the tone frequency) rises as the location of the current orientation comes closer to the predetermined orientation, with the goal being to achieve as high a tone as possible. The person conducting the procedure thus can, for example, initially locate the highest possible tone in a “top-to-bottom direction” in order, by a left-to-right movement of the instrument, to then also locate an optimal adjustment in this direction, wherein at this point a “switch back” to a different conversion scale of the deviation in the pitch can also be made for fine adjustment, so the highest tone must then be located again. This embodiment can be achieved particularly simply by a corresponding activation of the gradient coil arrangement, for example by a variation of the repetition time.

In a second specific embodiment of the method according to the invention, a tone repeating with a repetition frequency depending on the deviation is used as the acoustic signal, with the repetition frequency increasing with decreasing deviation, and a continuous tone is achieved given a deviation of zero. In this way it is possible to provide the acoustic feedback with regard to the orientation similar to a parking aid in a motor vehicle, which is already familiar to the person conducting the procedure. The repetition frequency of a tone thus increases continuously with decreasing distance until finally a continuous tone is achieved.

A continuous or cyclic acquisition of measurement data can take place to track the deviation of the current orientation from the predetermined orientation. In principle, a relatively fast measurement protocol is used to acquire the measurement data since it is only necessary to be able to recognize the current position of the instrument (or the instrument guide or an instrument substitute) in the acquired magnetic resonance images clearly enough in order to derive a current orientation from these. For example, acquisition times from 100-300 ms can be used. A cyclical acquisition of measurement data is suggested when the gradient coil arrangement is also used to emit the acoustic signal. A cyclical acquisition of measurement data then takes place, and activation of the gradient coil arrangement to emit the acoustic signal takes place in the data acquisition pauses. For example, the acquisition of measurement data can take place within a time period from 100-400 ms (preferably 200-300 ms), whereupon an acquisition pause of 600-1200 ms (preferably 700-800 ms) occurs in which an activation of the gradient coil arrangement can occur to emit the acoustic signal. The acquisition duration of the measurement duration and the duration of the acquisition pauses are preferably selected so as to have a total duration of a second. A new, current deviation can consequently be determined at one-second intervals.

To determine the current orientation using the measurement data, that two slice images can be acquired in slices parallel or orthogonal to one another, this measurement data showing (representing) the instrument and/or the instrument guide and/or an instrument substitute (visible in magnetic resonance images and used in place of the instrument in the instrument guide to align said instrument guide). Since the instrument and/or the instrument guide and/or the instrument substitute are visible in the images, the current orientation is determined from the localization thereof. For detection of the current orientation is to be conducted essentially in real time, an automatic instrument localization in magnetic resonance images acquired with a fast image acquisition technique is thus proposed. For example, it is possible to locate the instrument (for example the needle), the instrument guide or the instrument substitute in two slices images acquired in parallel planes, such that the direction of the instrument, and consequently its orientation in space, are known. However, projection-like magnetic resonance images are also conceivable. Thus, slice images are acquired in slices with a large thickness (encompassing the entire patient, for example), these slice images being perpendicular to one another, and the position and the orientation of the instrument can be determined in space from the respective projections. Such procedures are basically known and therefore do not need to be presented in detail herein.

As already indicated, it is important that the object that is detected in order to determine the current orientation of the instrument guide or the instrument in the measurement data of the magnetic resonance device be clearly visible in the measurement data. In a simple embodiment, an instrument provided with at least one marker that is visible in magnetic resonance images is used. For example, a tube that is visible in magnetic resonance, such as a tube filled with water, can be arranged around the instrument (the needle, for example), which is then easily detectable in the magnetic resonance images. It is also possible to provide the instrument guide with corresponding markers that are visible in magnetic resonance imaging.

Within the scope of the present invention, the use of an alignment device in which a set orientation of an instrument guide can be fixed, for example according to the manner of WO 2004/021898 A1 mentioned above, is advantageous. In this context, it can be advantageously to use an alignment device that may be fixed in a set orientation in order to align the instrument, and an instrument substitute that is visible in magnetic resonance images is inserted into the alignment device to adjust the predetermined orientation. After setting the predetermined orientation, the alignment device is fixed in this orientation and the instrument is inserted. An alignment device can thus be used as is commercially available under the name “SeeStar”, for example, which has an instrument guide that is adjustable in terms of its orientation, so a set orientation may be fixed. The system described in WO 2004/021898 A1 can be realized so as to be magnetic resonance-compatible and can be equipped with an instrument substitute that is visible in magnetic resonance images, for example a water-filled manual syringe. If the alignment of the instrument guide is adapted to the predetermined orientation with acoustic guidance and is fixed, the instrument substitute can be replaced by the actual instrument in order to finally conduct the procedure.

In a further embodiment of the present invention, a laser device is used to mark the predetermined entrance point and/or an area for the entrance point on the patient. For this purpose, a laser light source arranged outside of the patient receptacle can be used that produces a light point at the point of the predetermined procedure point.

In addition to the method, the invention also concerns a magnetic resonance device having a control device designed to implement the method according to the invention. In particular when the magnetic resonance device has, as a component thereof, an acoustic output emitter for communication with a patient and/or a gradient coil arrangement, these can be used as an acoustic output emitter for the acoustic signal so that additional devices are not necessary and the method according to the invention can proceed completely automatically in the control device, which then activates the corresponding acoustic output emitter as a component of the magnetic resonance device. The control device is then also designed (configured) to evaluate the measurement data. All embodiments with regard to the method according to the invention apply analogously to the magnetic resonance device according to the invention, with advantage comparable to the advantages already described above.

### BRIEF DESCRIPTION OF THE DRAWINGS

FIG. 1 schematically illustrates a magnetic resonance device according to the invention.

FIG. 2 is a flowchart of an embodiment of a procedure implemented with the method according to the invention.

FIG. 3 schematically illustrates an example of the determination of the current orientation that takes place in the inventive method.

### DESCRIPTION OF THE PREFERRED EMBODIMENTS

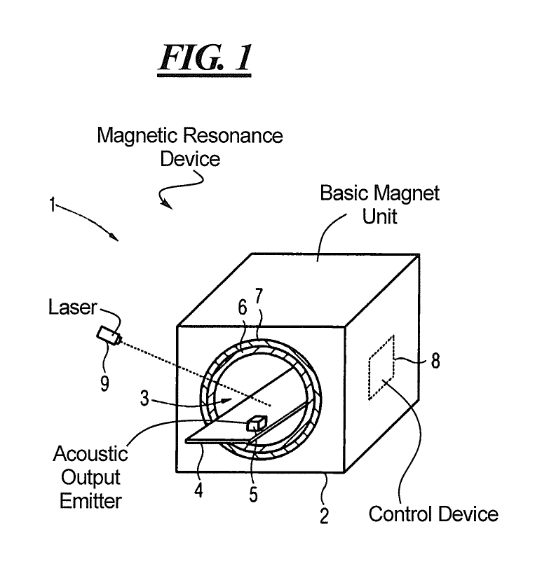

FIG. 1 shows a magnetic resonance device 1 according to the invention. As is generally known, the magnetic resonance device 1 has a basic magnet unit 2 that includes the superconducting coils to generate the basic magnetic field. A patient bed 4, which supports a patient to be examined and/or to be treated, can be driven into a patient receptacle 3 of the basic magnet unit 2. An acoustic output emitter 5 (which can be designed as headphones or a speaker) is provided for communication with the patient within the patient receptacle 3.

Additional components of the magnetic resonance device enclosing the patient receptacle 3 include a radio-frequency coil arrangement 6 and a gradient coil arrangement 7, the functioning of which in a typical MR data acquisition procedure is well known to those skilled in the art, and need not be presented herein.

The operation of the magnetic resonance device 1 is controlled by a central control device 8 that is also designed to implement the method according to the invention.

With the magnetic resonance device 1, k possible to implement minimally invasive procedures by means of an instrument (in the example a medical treatment needle) while the patient is located within the patient receptacle 3. In order to be able to mark a predetermined entrance point for the needle on the patient within the patient receptacle 3, a laser device 9 is provided that can be arranged outside of the patient receptacle 3, or even spatially within the patient receptacle 3, advantageously within the region of the isocenter. If the laser device 9 is provided within the patient receptacle 3, or if the patient bed 4 is located outside of the patient receptacle 3 at the point in time at which the entrance point is set or marked on the patient, an adjustment of the indicator on the patient can also take place by moving the patient bed 4.

The laser device does not need to exactly mark an entrance point; rather, it is also conceivable to mark an area—for example a good estimate—for the entrance point, and the actual desired entrance point can then be selected exactly in the area using real time imaging.

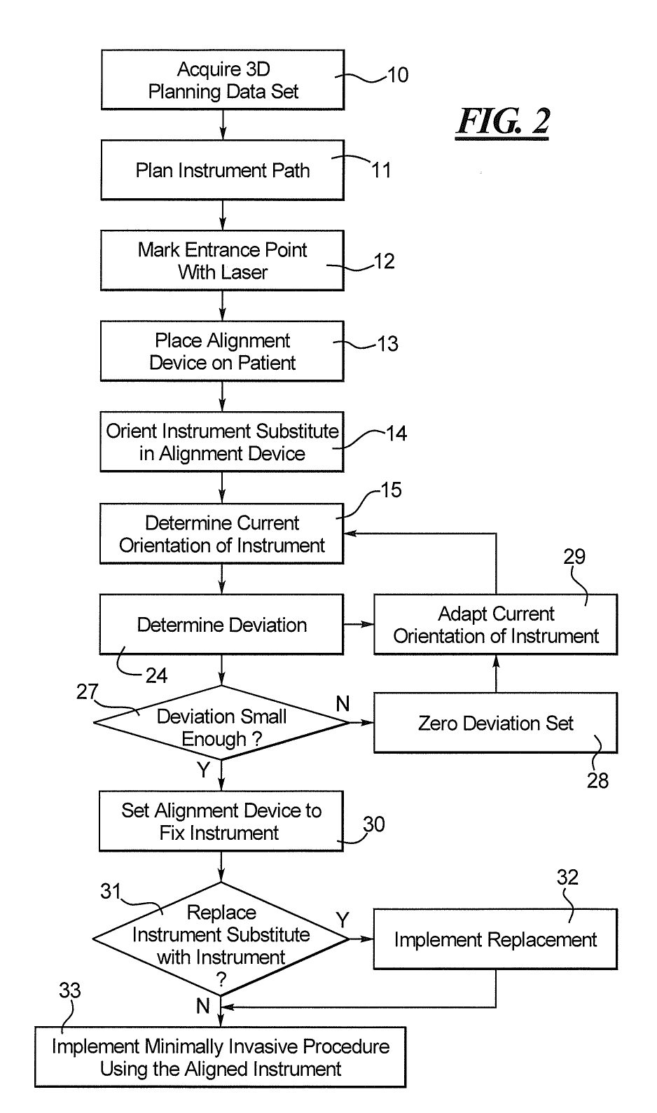

FIG. 2 shows basic steps in the preparation of the actual procedure, wherein an assistance to the person conducting the procedure takes place in the alignment of the needle by the method according to the invention, which method can run completely automatically in/with the control device 8.

The planning of the procedure precedes the actual procedure, for which a three-dimensional planning data set is acquired in Step 10. In Step 11, this is used in order to plan the trajectory or the path of the needle in advance. A desired entrance point and a desired entrance orientation inevitably result from this, and are designated as predetermined entrance point and predetermined orientation in the further description because they are the references according to which the actual placement of the instrument (thus the needle) is now aligned.

In order to assist the person conducting the procedure, in Step 12 the laser light point of the laser device 9 is caused to mark the predetermined entrance point on the patient. This can take place by adjustment of the laser device 9 and/or the patient bed 4.

In the exemplary embodiment shown here, a guide and alignment device is used as it is also described in WO 2004/021898 A1. In Step 13, this device is now placed on the patient so that the predetermined entrance point is established as an entrance point of a needle directed in the instrument guide of the alignment device, because in the alignment device that is used it is possible to arbitrarily change the orientation and instrument guide (and consequently the instrument to be guided) without varying the already established entrance point.

If the patient is not yet located in the patient receptacle 3, the patient can now be moved into the patient receptacle 3 by driven operation of the patient bed 4. In order to also now align the instrument guide so that the predetermined orientation is achieved for the instrument, the method according to the invention to assist the person conducted the procedure is implemented in the alignment of the instrument. Because the needle itself and the instrument guide of the alignment device are not visible in magnetic resonance images in the exemplary embodiment shown here, an instrument substitute is initially used in the instrument guide (Step 14). However, it is also possible to use an instrument that is visible anyway in magnetic resonance images, or to provide the instrument and/or the instrument guide with markers visible in magnetic resonance images, for example to use a water-filled jacket or the like for a needle.

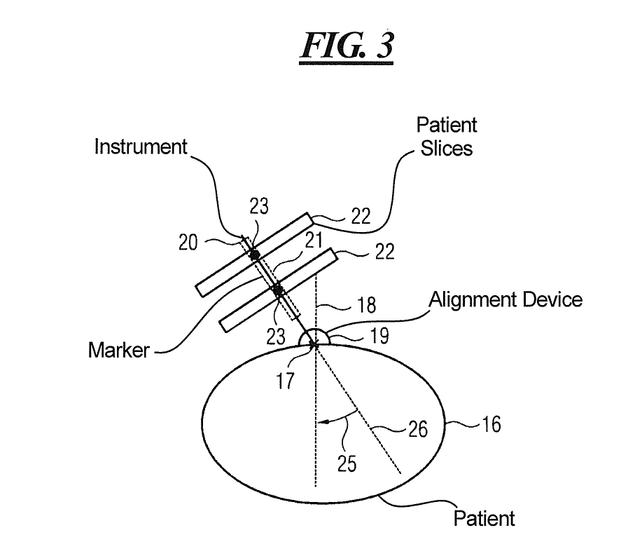

In Step 15 the current orientation of the instrument, the instrument guide or the instrument substitute is now determined from measurement data of the magnetic resonance device 1, which should be explained in detail via the presentation in FIG. 3. Schematically shown is the patient 16 on whose surface the predetermined entrance point 17 is located. The predetermined orientation is represented by a dashed line 18. The alignment device 19 is already placed on the patient 16, such that in each case the predetermined entrance point 17 is met by a needle (as instrument 20) inserted into its guide. The orientation of the instrument 20 (or of the instrument substitute) can be arbitrarily adjusted without the setting at the predetermined entrance point 17 being further changed. As shown in the example of FIG. 3, an instrument provided with a magnetic resonance marker can also be used in this step.

Two slice images in parallel slices 22 are now acquired by the magnetic resonance device 1, in which slice images the instrument 20 can be localized via the marker 21 (presently represented by the points 23) from which the orientation of the instrument can be derived in Step 15.

Naturally, the described procedure can also be transferred to an instrument substitute or an instrument guide—provided with corresponding markers—of the alignment device 19. Furthermore, alternative possibilities to determine the current orientation of the instrument 20 (or, respectively, of the instrument guide or of the instrument substitute) are naturally also conceivable, for example the acquisition of particularly thick, projection-like slices that are situated orthogonal to one another.

The deviation 25 of the current orientation of the instrument 20 (described by the dashed line 26) from the predetermination orientation (described by the dashed line 18) is then determined (as an angle in the example of FIG. 3) in Step 24 (FIG. 2).

In Step 27 it is then reviewed whether the deviation is small enough or whether no deviation is present at all, wherein in Step 28 an acoustic signal is output as acoustic feedback when a deviation differing from zero is present, wherein the acoustic signal depends on the determined deviation 25.

In the exemplary embodiment, the acoustic signal is emitted via the gradient coil arrangement 7 as a component of the magnetic resonance device 1. A measurement data acquisition is presently assumed in Step 15 that takes approximately 200 ms. During the acquisition of the measurement data, the gradient coil arrangement 8 is operated by the control device 8 corresponding to the necessary measurement protocol. However, afterwards it is used to emit the acoustic signal, presently for a duration of 800 ms. For this purpose, in the activation of the gradient coil arrangement 7, the repetition time is varied depending on the deviation 25 such that a tone of a specific pitch is created as an acoustic signal via the interaction of the gradient coil arrangement 7 with structural components of the magnetic resonance device 1, with the pitch then accordingly depending on the deviation 25 and the tone and becoming increasingly higher with decreasing deviation 25. For example, repetition times of 5 ms (tone frequency 200 Hz) to 1.5 ms (tone frequency 700 Hz) can be worked with. An acquisition of measurement data (Step 15) thus takes place cyclically, wherein the gradient coil arrangement 7 is activated in the acquisition pauses to output the acoustic signal.

There are naturally other variants to emit the acoustic signal, such that the acoustic signal can be generated via the acoustic output emitter 5, for example when differentiated acoustic signals are desired. It is also conceivable to generate an otherwise intuitive guidance via emission of the acoustic signal, such that achieving a maximum tone level does not necessarily need to represent the goal for the person conducting the procedure. For example, a tone with a specific repetition frequency can be reproduced that increases with approach to the predetermined orientation, such that ultimately a continuous tone results upon agreement (as is known from parking aids in motor vehicles). An extremely intuitive guidance of the person conducting the procedure can hereby also take place.

For the person conducting the procedure, in Step 29 the possibility consequently exists to adapt the current orientation of the instrument 20/of the instrument guide/of the instrument substitute so that the deviation 25 (and thus also the acoustic signal) changes. For example, the person conducting the procedure can optimize the alignment only in a “top-to-bottom direction” in order to then achieve an optimization in the “left-to-right direction”.

If it is now established (Step 27) that the predetermined orientation is achieved, in Step 30 the set orientation at the alignment device 19 is fixed.

In Step 31 a check is made as to whether an instrument substitute was used instead of the instrument 20. If yes, in Step 32 the instrument substitute is replaced by the instrument 20. Finally, the minimally invasive procedure can take place in Step 33.

For a more precise adjustment of the predetermined orientation, this can be implemented in multiple stages by, upon falling below a limit value for the deviation 25, a transition can be made to a different dependency of the acoustic signal on the deviation 25 (in particular in the sense of a scaling) so that a fine adjustment can subsequently take place.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method to provide assistance in alignment of an instrument in a minimally invasive medical procedure conducted on a patient located within a patient receptacle of a magnetic resonance device, said instrument to be introduced into the patient at an entrance point with a predetermined orientation, said method comprising the steps of:
operating said magnetic resonance device to acquire magnetic resonance measurement data, and reconstructing an image from said measurement data, in which a current orientation of said instrument or an instrument guide or a substitute instrument is represented;
in a processor, automatically determining, from said magnetic resonance image, a current orientation of said instrument or said instrument guide or said substitute instrument;
in said processor, automatically determining a deviation of said current orientation from said predetermined orientation; and
from said processor, causing an acoustic signal to be emitted that is dependent on said deviation.

2. A method as claimed in claim 1 wherein said magnetic resonance device comprises a magnetic resonance device component that is capable of acoustic emission, and comprising, from said processor, operating said component to emit said acoustic signal.

3. A method as claimed in claim 2 comprising using an acoustic emitter configured for audio communication with a patient in said magnetic resonance device as said component.

4. A method as claimed in claim 2 comprising using a gradient coil arrangement of said magnetic resonance device as said component.

5. A method as claimed in claim 4 comprising operating said gradient coil arrangement to emit noise with a repetition time dependent on said deviation in order to generate said acoustic signal.

6. A method as claimed in claim 1 comprising emitting, as said acoustic signal, a tone having a tone characteristic selected from the group consisting of repetition frequency and pitch, and, from said processor, changing said tone characteristic dependent on said deviation.

7. A method as claimed in claim 1 comprising, from said processor, operating a gradient coil arrangement of said magnetic resonance device to generate a tone having a pitch, as said acoustic signal, and, from said processor, changing said pitch of said tone dependent on said deviation, with a maximum value of said pitch, selected from the group consisting of a highest pitch and a lowest pitch, representing a deviation of zero.

8. A method as claimed in claim 1 comprising, from said processor, causing a tone to be emitted, as said acoustic signal, with a repetition frequency dependent on said deviation, said repetition frequency increasing with decreasing deviation and emitting a continuous tone to represent a deviation of zero.

9. A method as claimed in claim 1 comprising operating said magnetic resonance device to acquire said MR measurement data as two slice images representing slices of the patient that are situated parallel or orthogonal to each other, and determining said current orientation from said two slice images.

10. A method as claimed in claim 9 comprising determining said current orientation from said two slice images by, in said processor, localizing the representation of said instrument or said instrument guide or said instrument substitute in said two slice images.

11. A method as claimed in claim 1 comprising providing said instrument with a magnetic resonance-visible marker, and using a visible indication of said marker in said magnetic resonance image as said representation of said instrument.

12. A method as claimed in claim 1 comprising mechanically aligning said instrument relative to said entrance point using an alignment device in which said instrument or said instrument guide or said instrument substitute is adjustable to an orientation in said alignment device, and setting said instrument or said instrument guide or said instrument substitute in said alignment device initially in said predetermined orientation.

13. A method as claimed in claim 1 comprising using a laser device to provide an indication on the patient coinciding with said entrance point or a limited area surrounding said entrance point.

14. A method as claimed in claim 1 comprising tracking said deviation of said current orientation from said predetermined orientation with a cyclical acquisition of said MR measurement data.

15. A method as claimed in claim 14 comprising implementing said cyclical acquisition of said MR measurement data while using a gradient coil arrangement of said magnetic resonance device to emit said acoustic signal, and, from said processor, activating said gradient coil arrangement to emit said acoustic signal in pauses between said cyclical acquisition of said magnetic resonance measurement data.

16. A magnetic resonance apparatus to provide assistance in alignment of an instrument in a minimally invasive medical procedure comprising:
a magnetic resonance data acquisition unit having a patient receptacle therein configured to receive a patient therein to allow a minimally invasive procedure to be conducted on the patient located within the patient receptacle, said procedure involving an instrument to be introduced into the patient at an entrance point with a predetermined orientation;
a control unit configured to operate said magnetic resonance data acquisition unit to acquire magnetic resonance measurement data;
a processor configured to reconstruct an image from said measurement data, in which a current orientation of said instrument or an instrument guide or a substitute instrument is represented;
said processor being configured to automatically determine, from said magnetic resonance image, a current orientation of said instrument or said instrument guide or said substitute instrument;
said processor being configured to automatically determine a deviation of said current orientation from said predetermined orientation; and
said processor being configured to cause an acoustic signal to be emitted that is dependent on said deviation.

17. A magnetic resonance apparatus as claimed in claim 16 wherein said magnetic resonance data acquisition unit comprises gradient coils, and wherein said control unit is configured to operate said gradient coils to participate in the acquisition of said MR measurement data, and wherein said processor is configured to cause said control unit to also operate said gradient coils to emit said acoustic signal.

18. A magnetic resonance apparatus as claimed in claim 17 wherein said control unit is configured to operate said MR data acquisition unit to cyclically acquire said MR measurement data, and wherein said processor is configured to cause said control unit to operate said gradient coils to emit said acoustic signal in pauses between the cyclical acquisition of said MR measurement data.
